(19) United States
(12) Patent Application Publication
(10) Pub. No.: US 2024/0349821 A1
(43) Pub. Date: Oct. 24, 2024

(54) AEROSOL GENERATING DEVICE WITH HEATING

(71) Applicant: Philip Morris Products S.A., Neuchatel (CH)

(72) Inventors: Yves JORDIL, Lausanne (CH); Jennifer STUECKER, Bellerive (CH)

(73) Assignee: Philip Morris Products S.A., Neuchatel (CH)

(21) Appl. No.: 18/686,205

(22) PCT Filed: Aug. 26, 2022
(86) PCT No.: PCT/EP2022/073858

§ 371 Date: Feb. 23, 2024

(30) Foreign Application Priority Data
Sep. 3, 2021 (EP) 21194810.4

(51) Int. Cl. A24F 42/10; A24F 7/00; A24F 42/60; F24V 30/00

(52) U.S. Cl. CPC A24F 42/10; A24F 7/00; A24F 42/60; F24V 30/00

(57) ABSTRACT

An aerosol-generating system is provided, including: an aerosol-generating device including a mouthpiece, a heating chamber, at least one heating article disposed in the heating chamber, a consumable chamber in fluid communication with the mouthpiece, the consumable chamber including an opening and a closure movable between an open position in which a plurality of aerosol-generating elements may be inserted into or removed from the consumable chamber through the opening, and a closed position in which the plurality of aerosol-generating elements are retained in the consumable chamber, and a plurality of aerosol-generating elements disposed in the consumable chamber. An aerosol-generating device, and a kit of parts including the aerosol-generating device, are also provided.

The present invention relates to an aerosol-generating device. In particular, the present invention relates to an aerosol-generating device for use with at least one heating article and at least one aerosol-generating element. The present invention also relates to an aerosol-generating system comprising the aerosol-generating device, at least one heating article and at least one aerosol-generating element. The present invention also relates to a kit of parts comprising the aerosol-generating device, at least one heating article and at least one aerosol-generating element.

Aerosol-generating articles in which an aerosol-generating substrate, such as a nicotine-containing substrate or a tobacco-containing substrate, is heated rather than combusted, are known in the art. Typically, in such heated smoking articles an aerosol is generated by the transfer of heat from a heat source to a physically separate aerosol-generating substrate or material, which may be located in contact with, within, around, or downstream of the heat source. During use of the aerosol-generating article, volatile compounds are released from the aerosol-generating substrate by heat transfer from the heat source and are entrained in air drawn through the aerosol-generating article. As the released compounds cool, they condense to form an aerosol. Substrates for heated aerosol-generating articles have, in the past, often been produced using randomly oriented shreds, strands, or strips of tobacco material.

Aerosol-generating devices for consuming such aerosol-generating articles are also known in the art. Such devices include, for example, electrically heated aerosol-generating devices in which an aerosol is generated by the transfer of heat from one or more electrical heater elements of the aerosol-generating device to the aerosol-generating substrate of a heated aerosol-generating article.

However, such aerosol-generating devices typically include an electrical storage means such as batteries or capacitors. These electrical storage means require charging from an external power source which is typically a mains electricity supply. The mains electricity supply may not be generated from a renewable source such as wind turbines or solar cells. In addition, if the electrical storage means is fully depleted and a user is not able to use an external power supply, then the user may be unable to use the aerosol-generating device at all, limiting the aerosol-generating device's use.

In addition, in aerosol-generating devices of the prior art, a user has a limited ability to customise their user experience. In particular, a user may want to customise the flavour delivered by the aerosol-generating article, the duration of the user experience, and the amount of aerosol delivered. While a user may select different aerosol-generating articles for use with the aerosol-generating device, this is the limit of how customisable a user experience can be.

Accordingly, there is a need to provide an aerosol-generating device which provides a more sustainable means to generate an aerosol. There is also a need to provide an aerosol-generating device which facilitates customisation of the user experience.

The present disclosure relates to an aerosol-generating device. The aerosol-generating device may comprise a mouthpiece. The aerosol-generating device may comprise a heating chamber for receiving at least one heating article. The aerosol-generating device may comprise a consumable chamber for receiving at least one aerosol-generating element. The consumable chamber may be in fluid communication with the mouthpiece. The consumable chamber may comprise an opening. The consumable chamber may comprise a closure movable between an open position in which the at least one aerosol-generating element may be inserted into or removed from the consumable chamber through the opening, and a closed position in which the at least one aerosol-generating element is retained in the consumable chamber.

According to the present invention, there is provided an aerosol-generating device comprising a mouthpiece, a heating chamber for receiving at least one heating article, and a consumable chamber for receiving at least one aerosol-generating element. The consumable chamber is in fluid communication with the mouthpiece, the consumable chamber comprising an opening and a closure movable between an open position in which the at least one aerosol-generating element may be inserted into or removed from the consumable chamber through the opening, and a closed position in which the at least one aerosol-generating element is retained in the consumable chamber.

The aerosol-generating device of the present invention may be used in combination with at least one heating article and at least one aerosol-generating element. In use, at least one heating article may be placed in the heating chamber and at least one aerosol-generating element may be placed in the consumable chamber. The at least one heating article may be activated and heat generated by the at least one heating article may heat the at least one aerosol-generating element in the consumable chamber. The heated aerosol-generating element may generate or otherwise emit an aerosol in the consumable chamber. The generated aerosol may leave the aerosol-generating device through the mouthpiece.

The provision of a consumable chamber for receiving the at least one aerosol-generating element may advantageously allow a user to customise their user experience by selecting different aerosol-generating elements for use in the device. Where more than one aerosol-generating element may be received in the consumable chamber at a time, a user may be able to further customise their user experience by varying the specific combination of different aerosol-generating elements or by varying how the aerosol-generating elements are arranged or ordered in the consumable chamber.

The provision of a heating chamber for receiving at least one heating article may advantageously further allow a user to customise their user experience. In aerosol-generating devices of the prior art which include an integral electrical heater, a user may be unable to vary the temperature of the heater, or may only be able to vary the temperature to a limited extent. In the present invention, a user may insert different heating articles into the heating chamber to customise their experience to a greater extent than is possible in prior art devices. For example, a user may be able to choose between different heating articles which operate at different temperatures. A user may be able to select different heating articles which provide different temperature profiles. Where a specific aerosol-generating element is intended to be heated to a specific temperature or using a specific heating profile, a user may advantageously be able to select an appropriate heating article for use with a corresponding aerosol-generating element.

Where more than one heating article may be received in the heating chamber at a time, a user may be able to further customise their user experience by varying the specific combination of different heating articles or by varying how the heating articles are arranged or ordered in the heating chamber.

Since the aerosol-generating device generates heat by using separate heating articles, the aerosol-generating device may not need to include any electronic components such as electrical heaters or batteries. This may advantageously simplify the manufacture of the aerosol-generating device. In addition, this may advantageously eliminate the need for the aerosol-generating device to require charging from an external power source.

As used herein, the term “aerosol-generating element” refers to a discrete aerosol-generating substrate. The aerosol-generating element may be solid. The aerosol-generating element may comprise tobacco, for example homogenised tobacco. The aerosol-generating element may comprise an aerosol former.

The aerosol-generating element may comprise an aerosol-generating formulation dispersed and encapsulated within a cross-linked polymer matrix. The structure and composition of the at least one aerosol-generating element will be described in more detail below.

As used herein, the term “aerosol-generating substrate” refers to a substrate capable of releasing upon heating volatile compounds, which can form an aerosol. The aerosol generated from the at least one aerosol-generating element described herein is a dispersion of solid particles or liquid droplets (or a combination of solid particles and liquid droplets) in a gas. The aerosol may be visible or invisible and may include vapours of substances that are ordinarily liquid or solid at room temperature as well as solid particles or liquid droplets or a combination of solid particles and liquid droplets.

As used herein, the term “heating article” refers to a discrete component which generates heat. The at least one heating article may require activation in order to initiate heat generation. The at least one heating article may generate heat by any means. The at least one heating element may generate heat by using an electrical heating element. Preferably, as discussed below the at least one heating article may generate heat by an exothermic chemical or physical change.

The heating chamber and the consumable chamber may be isolated from each other such that heating chamber is not in fluid communication with the consumable chamber.

Where this is the case, any substances in the consumable chamber, such as a generated aerosol, are not able to enter the heating chamber. This may advantageously prevent any substances from the consumable chamber interfering with the at least one heating article which may affect its ability to generate heat.

Similarly, any substances in the heating chamber, such as exhaust gases from a chemical reaction, are not able to enter the consumable chamber. This may advantageously prevent any substances from the heating chamber from interfering with aerosol generation, or possibly from being inhaled by a user.

The consumable chamber may comprise an air inlet. The air inlet may allow air to flow into the consumable chamber where it may become entrained with the generated aerosol before leaving the aerosol-generating device through the mouthpiece. This may also advantageously provide an acceptable resistance to draw for a user.

The heating chamber may include an opening such that at least one heating article may be inserted into and removed from the heating chamber.

The provision of an opening in the heating chamber may allow a user to remove used heating articles and to replace them with new, unused heating articles. This may be advantageous where the at least one heating article is single, or limited, use and require replacing or recharging after a specific number of user experiences. The provision of an opening may also advantageously allow a user to easily customise their experience by choosing to insert different heating articles into the heating chamber.

The closure of the consumable chamber may also close the opening of the heating chamber, such that when the closure is in the open position at least one heating article may be inserted into or removed from the heating chamber, and when the closure is in the closed position, the at least one heating article is retained in the heating chamber.

The provision of a closure closing the opening of the heating chamber may advantageously prevent the at least one heating article from falling out of the aerosol-generating device in use. The provision of the closure closing both the opening of the heating chamber and the opening of the consumable chamber may mean that both chambers may be opened and closed by a user at the same time. This may advantageously make preparing the aerosol-generating device for use more convenient, particularly where the at least one aerosol-generating element and heating article both require replacing between uses.

The closure of the consumable chamber may be located adjacent the opening of the heating chamber. This may advantageously enable the closure to be as compact as possible.

The closure may be fully detachable from the remainder of the aerosol-generating device. Where this is the case, the closure may be fully detached from the remainder of the aerosol-generating device when the closure is in the open position.

The closure may be attached to the remainder of the aerosol-generating device in both the open and closed positions. Where this is the case, the closure may be attached to the remainder of the aerosol-generating device by a hinge, a pin, or a resiliently deformable member.

The closure may include a retaining means for securing the closure in the closed position. This may advantageously prevent the closure opening inadvertently. The retaining means may include a latch, a catch, or an interference fit.

The closure may comprise the mouthpiece.

Where this is the case, the closure may comprise an airflow passage which is in fluid communication with the opening of the consumable chamber. The airflow passage may be sized to prevent the at least one aerosol-generating element from passing through the airflow passage when the closure is in the closed position such that the closure is still able to securely retain the at least one aerosol-generating element.

The provision of the closure comprising the mouthpiece may advantageously offer a convenient solution for providing both features thereby preventing the need to include a closure and a separate mouthpiece. This provision may also allow the opening of the consumable chamber to provide both access for removing and replacing the at least one aerosol-generating element, but also the means by which the aerosol may leave the consumable chamber through the mouthpiece. This may minimise the number of openings in the consumable chamber which may advantageously make manufacturing more straightforward and may improve the strength of the aerosol-generating device.

Where the closure comprises the mouthpiece, a portion of the closure fully overlies the opening of the heating chamber when the closure is in the closed position to substantially prevent air entering or leaving the heating chamber through the mouthpiece. This may advantageously prevent any exhaust gases from the at least one heating article from entering the mouthpiece and being inhaled by a user.

The aerosol-generating device may further comprise a heat-conducting element disposed between the heating chamber and the consumable chamber.

The provision of a heat-conducting element between the heating chamber and the consumable chamber may improve heat transfer from the at least one heating article to the at least one aerosol-generating element. This may advantageously improve aerosol generation from the at least one aerosol-generating element disposed in the consumable chamber.

The heat-conducting element may comprise any material. The heat-conducting element may comprise a metallic material. Suitable heat-conducting elements for use in the present invention include, but are not limited to, aluminium, steel, iron, copper, and alloys thereof. The heat-conducting element may comprise a metallic foil material.

As used herein, the term “heat-conducting material” is used to describe a material having a bulk thermal conductivity of at least about 10 W per metre Kelvin (W/(m·K)) at 23° C. and a relative humidity of 50% as measured using the modified transient plane source (MTPS) method.

The aerosol-generating device may comprise an external housing. The external housing may be heat insulating.

The provision of a heat insulating external housing may advantageously prevent the outer surface of the aerosol-generating device from becoming too hot. This provision may also retain the maximum amount of heat inside the aerosol-generating device. This may advantageously improve the generation of aerosol from the at least one aerosol-generating element in the consumable housing.

The external housing may comprise a heat insulating material. The heat insulating material may be any heat insulating material. For example, the heat insulating material may by a polymeric material. The polymeric material may include one or more of polyaryletherketone (PAEK), polyether ether ketone (PEEK), and polyphenylene sulfone (PPSU).

The external housing may comprise a heat insulating construction. For example, the external housing may comprise a double wall structure. The double wall structure may comprise air, foam, or a vacuum between two walls to provide insulation.

As used herein, the term “heat insulating” is used to describe a material or a construction having a thermal conductivity of less than about 50 milliwatts per metre Kelvin (mW/(m·K)) at 23° C. and a relative humidity of 50% as measured using the modified transient plane source (MTPS) method.

The opening of the consumable chamber may be located at the upstream end of the consumable chamber. The opening of the consumable chamber may be located on a longitudinal surface of the consumable chamber.

The opening of the consumable chamber may be located at the downstream end of the consumable chamber.

Locating the opening at the downstream end of the consumable chamber may be particularly advantageous where the closure comprises the mouthpiece. Where the closure comprises the mouthpiece, providing the opening of the consumable chamber at the downstream end is advantageous since it locates the opening near the mouthpiece such that the airflow passage of the mouthpiece may readily be in fluid communication with the opening of the consumable passage when the closure is in the closed position.

The opening of the heating chamber may be located at the upstream end of the heating chamber. The opening of the heating chamber may be located on a longitudinal surface of the heating chamber.

The opening of the heating chamber may be located at the downstream end of the heating chamber.

Locating the opening at the downstream end of the heating chamber may be particularly advantageous where the closure comprises the mouthpiece. Where the closure comprises the mouthpiece, providing the opening of the heating chamber at the downstream end is advantageous since it enables the closure to readily close the opening of the heating chamber when the closure is in the closed position.

As used herein, the terms “upstream” and “downstream”, are used to describe the relative positions of components, or portions of components, of the aerosol-generating device, or aerosol-generating system in relation to the direction in which air flows through the aerosol-generating device during use thereof. Components, or portions of components, of the aerosol-generating device, or aerosol-generating system may be described as being upstream or downstream of one another based on their relative positions between an upstream end of the aerosol-generating device, or aerosol-generating system and a downstream end of the aerosol-generating device, or aerosol-generating system.

As used herein, the term “longitudinal axis” refers to the axis extending between the upstream end of the aerosol-generating device and the downstream end of the aerosol-generating device. A longitudinal direction is a direction parallel to the longitudinal axis of the aerosol-generating device and a longitudinal surface of the heating chamber is a surface of the heating chamber which extends between the upstream end of the heating chamber and the downstream end of the heating chamber.

The heating chamber may comprise a longitudinal opening in which the consumable chamber is disposed.

Where this is the case, the consumable chamber may be fully surrounded along its longitudinal length by the heating chamber. This may ensure that heat is provided to the consumable chamber from all sides of the consumable chamber. This may advantageously ensure efficient heating of the at least one aerosol-generating element during use of the aerosol-generating device.

The heating chamber may be any shape. The heating chamber may be generally cylindrical including an upstream face, a downstream face, and a longitudinal surface extending between the upstream face and the downstream face.

The heating chamber may be toroidal in shape. The heating chamber may be a toroidal cylinder. In other words, the heating chamber may be generally cylindrical in shape and include an opening which passes from the upstream face to the downstream face. The opening may be disposed along the longitudinal axis of the heating chamber. Where the heating chamber is toroidal in shape, the opening of the heating chamber through which heating articles may be inserted into or removed may also be toroidal in shape.

The consumable chamber may comprise a longitudinal opening in which the heating chamber is disposed.

Where this is the case, the heating chamber may be fully surrounded along its longitudinal length by the consumable chamber. This may provide the greatest distance between the heating chamber and the external surface of the aerosol-generating device. This may advantageously prevent the external surface of the aerosol-generating device from becoming too hot. This may also advantageously provide efficient heating of the consumable chamber by the at least one heating article disposed in the heating chamber.

The consumable chamber may be generally cylindrical including an upstream face, a downstream face, and a longitudinal surface extending between the upstream face and the downstream face.

The consumable chamber may be toroidal in shape. The consumable chamber may be a toroidal cylinder. In other words, the consumable chamber may be generally cylindrical in shape and include an opening which passes from the upstream face to the downstream face. The opening may be disposed along the longitudinal axis of the consumable chamber.

The heating chamber may be formed from a resiliently deformable material.

The resiliently deformable material may include a polymeric material. Examples of suitable resiliently deformable polymeric materials include, polypropylene, polyethylene, polystyrene, polyethylene terephthalate, acrylonitrile butadiene styrene, and combinations thereof.

The provision of a heating chamber formed from a resiliently deformable material may allow a user to mechanically deform the at least one heating article disposed in the heating chamber. Where the at least one heating article requires mechanical deformation in order to activate the heat generation, the provision of a resiliently deformable material may advantageously allow a user to activate the at least one heating article without needed to directly access the at least one heating article. For example, where the at least one heating article comprises a heating article in which heat is generated by an exothermic chemical or physical change, a user may be able to ‘crack’ the at least one heating article to initiate the exothermic chemical or physical change by squeezing the resiliently deformable heating chamber.

The heating chamber may be configured to receive at least one heating article which is configured to generate heat by an exothermic chemical or physical change.

The provision of a heating chamber configured to receive heating article which is configured to generate heat by an exothermic chemical or physical change may allow for the aerosol-generating device to generate heat without the need for any electronic components such as electrical heaters or batteries. This may advantageously mean that the aerosol-generating device does not need to be charged from an external power source which may not be generated from a renewable source.

The at least one heating article may be one in which heat is generated by an exothermic chemical change. Where this is the case, two or more chemical reagents may combine and chemically react to form one or more reaction products, the reaction generating heat. Alternatively or in addition, a single reagent may decompose to form a plurality of reaction products, the reaction generating heat.

The at least one heating article may be one in which heat is generated by an exothermic physical change. The physical change may involve one or more substances changing from a liquid state to a solid state, or from a solid state to a liquid state. Alternatively or in addition, the physical change may involve one or more substances changing from a first solid state to a second solid state, or from a first liquid state to a second liquid state. The at least one heating article may comprise a phase change material.

As used herein, the term “phase change material” refers to a substance which releases an appreciable amount of energy during a phase transition. The phase change material may have a first phase at a high energy state and a second phase at a lower energy state. When the phase change material moves from the high energy state to the lower energy state, useful energy is released. This energy is used to heat the at least one aerosol-generating element disposed in the consumable chamber. Further details of the at least one heating article are discussed below.

The heating chamber may be configured to receive at least one heating article which generates heat without electrical power.

This may mean that the aerosol-generating device does not include an electrical heater or an associated internal power source to power the electrical heater. This may advantageously mean that the aerosol-generating device does not need to be charged from an external power source which may not be generated from a renewable source. As set out above, the at least one heating article may generate heat by an exothermic chemical or physical change.

In addition, this may mean that the heating chamber does not require wires or other electrical contacts to project into the heating chamber. This may advantageously help to maintain the integrity of the heating chamber by minimising the number of holes projecting into the heating chamber.

The aerosol-generating device may include no electrical components. Alternatively, the aerosol-generating device may still include electrical components which provide functions other than generating heat. For example, the aerosol-generating device may include a user interface, a puff counter, an inhalation volume detector, or a temperature sensor.

The heating chamber may be divided into a first heating chamber section and a second heating chamber section by a heating chamber barrier, such that a first heating article disposed in the first heating chamber section is kept apart from a second heating article disposed in the second heating chamber section.

This arrangement may be particularly advantageous where the aerosol-generating device is intended to be used with at least one heating article comprising a first heating article comprising a first reagent and a second heating article comprising a second reagent, which react together to generate heat in an exothermic reaction. The first heating article comprising a first reagent may be disposed in the first heating chamber section and the second heating article comprising a second reagent may be disposed in the second heating chamber section. This may advantageously prevent the first and second reagents from reacting prematurely.

The heating chamber barrier may be movable between a closed position in which the first heating chamber section and a second heating chamber section are separated such that a first heating article disposed in the first heating chamber section is kept apart from a second heating article disposed in the second heating chamber section, and an open position in which the first heating article disposed in the first heating chamber section is able to mix with the second heating article disposed in the second heating chamber section.

This arrangement may advantageously allow a first heating article comprising a first reagent disposed in the first heating chamber section, and a second heating article comprising a second reagent disposed in the second heating chamber section to be kept separate until such time as heat generation is needed. When heat is required from the at least one heating article, the user moves the heating chamber barrier from the closed position to the open position, allowing the first heating article disposed in the first heating chamber section to mix with the second heating article disposed in the second heating chamber section. The combination of the first heating article and the second heating article may generate heat by an exothermic chemical or physical change between reagents disposed in the first heating article or second heating article.

The provision of the movable barrier may advantageously allow a greater range of different heating articles to be used with the aerosol-generating device.

The heating chamber barrier may be formed from a frangible material which, when broken, allows the first heating article disposed in the first heating chamber section to mix with the second heating article disposed in the second heating chamber section. A user may break the frangible barrier by squeezing or bending the heating chamber. A user may break the frangible barrier by actuating a control on the aerosol-generating device which breaks the frangible barrier.

The aerosol-generating device may further comprise an electrical power source and electrical circuitry configured to initiate an exothermic chemical or physical change in at least one heating article.

Some exothermic chemical or physical changes require a certain activation energy to initiate the chemical or physical change. Where this is the case, the energy may be provided by an electrical power source which initiates the chemical or physical change. For example, the electrical power source may be connected to a heating element which is configured to provide just enough heat to the at least one heating article to initiate the chemical or physical change. Alternatively, or in addition, the electrical power source may be connected to electrodes which are configured to apply an electric current directly to the at least one heating element to initiate the chemical or physical change. Once the chemical or physical change has been initiated, the electrical power source and electrical circuitry may be shut off and all of the energy used to heat the at least one aerosol-generating element may come from the exothermic chemical or physical change of the at least one heating article. Advantageously, no more electrical energy is provided than that which is needed to initiate the chemical or physical change.

The electrical power source may be any electrical power source. The electrical power source may be at least one of a battery, a capacitor, or a supercapacitor. The electrical power source may be rechargeable from an external power source.

The electrical circuitry may comprise a heating element configured to heat at least one heating article disposed in the heating chamber.

The heating element may be disposed in or near the heating chamber. The electrical circuitry may include a user interface element by which a user may activate the electrical circuitry to initiate the exothermic chemical or physical change in the at least one heating article. The electrical circuitry may comprise a controller to control the electrical power supplied from the electrical power source to other elements of the control circuitry.

The heating chamber may comprise a gas outlet.

Where the aerosol-generating device is to be used with at least one heating article configured to generate heat by an exothermic chemical change, the exothermic chemical change may release an exhaust gas as a reaction product. For example, where the exothermic chemical change includes the combustion of a carbonaceous material, carbon dioxide gas will be released. The provision of a gas outlet allows any exhaust gas to be released from the heating chamber. This may advantageously prevent the pressure in the heating chamber increasing to a level which may damage the aerosol-generating device. This may also prevent waste gases from stifling or otherwise retarding the chemical change occurring in the combustion chamber.

The gas outlet may be smaller than the heating chamber outlet. This may advantageously prevent the at least one heating article form passing through the gas outlet.

The gas outlet may be disposed in the upstream half of the heating chamber. The gas outlet may be disposed at the upstream end of the heating chamber. This may ensure that the exhaust gases are released as far from a user as possible during use of the aerosol-generating device. This may advantageously limit the amount of exhaust gas inhaled by the user.

According to the present invention, there is also provided an aerosol-generating system comprising, an aerosol-generating device as described above, at least one heating article disposed in the heating chamber, and at least one aerosol-generating element disposed in the consumable chamber.

According to the present invention, there is also provided a kit of parts comprising, an aerosol-generating device as described above, at least one heating article sized to be received in the heating chamber, and at least one aerosol-generating element sized to be received in the consumable chamber.

The at least one aerosol-generating element may have any shape. The at least one aerosol-generating element may have the shape of a bead, capsule, plug, or tablet.

The at least one aerosol-generating element may have a shape in the form of at least one of a sphere, a cylinder, or a toroid.

The at least one aerosol-generating element may have a cross section shape which corresponds to the cross sectional shape of the consumable chamber. This may allow the at least one aerosol-generating element to be securely retained in the consumable chamber such that it cannot move around within the chamber. This may advantageously improve heat transfer from the at least one heating article to the at least one aerosol-generating element since it minimises air pockets within the consumable chamber. This may also prevent multiple aerosol-generating elements from moving past each other or becoming mixed up inside the consumable chamber. This may be advantageous where a user wishes to achieve a customised user experience by arranging a plurality of different aerosol-generating elements in a specific order. This may provide a unique flavour for the user, or may provide a unique series of flavours which are generated one after the other.

Where the at least one aerosol-generating element is spherical or cylindrical in shape, the consumable chamber may be generally cylindrical and may have a diameter which is slightly larger than the diameter of the at least one aerosol-generating element. Where the at least one aerosol-generating element is toroidal in shape, the consumable chamber may also have the general shape of a toroid. Where the consumable chamber is configured to receive more than one aerosol-generating element, the consumable chamber may be a toroidal cylinder.

The at least one aerosol-generating element may have an equivalent diameter of at least about 0.5 millimetres.

The term “equivalent diameter” is used herein to denote the diameter of the sphere which has the same volume as the aerosol-generating element, regardless of the shape of the at least one aerosol-generating element. As described above, the at least one aerosol-generating element may have any shape. For an aerosol-generating element having a spherical shape and a circular transverse cross-section, the equivalent diameter is the diameter of the cross-section of the at least one aerosol-generating element.

The at least one aerosol-generating element may have an equivalent diameter of at least about 1 millimetre, at least about 2 millimetres, or at least about 3 millimetres.

The at least one aerosol-generating element may have an equivalent diameter of less than or equal to about 8 millimetres, less than or equal to about 6 millimetres, or less than or equal to about 5 millimetres.

The at least one aerosol-generating element may have an equivalent diameter from about 0.5 millimetres to about 8 millimetres, from about 1 millimetre to about 8 millimetres, from about 2 millimetres to about 8 millimetres, or from about 3 millimetres to about 8 millimetres.

The at least one aerosol-generating element may have an equivalent diameter of about 4 millimetres or about 4.5 millimetres.

The at least one aerosol-generating element may have a larger equivalent diameter. For example, the at least one aerosol-generating element may have an equivalent diameter or at least about 5 millimetres, at least about 7 millimetres, or at least about 10 millimetres.

The consumable chamber may have a height which is n times the height of a single aerosol-generating element, where n is an integer. For example, the consumable chamber may have a height which is 1, 2, 3, 4, 5, or 6 times the height of a single aerosol-generating element. This provision prevents any excess space in the consumable chamber when the consumable chamber is full of aerosol-generating elements. This advantageously provides more efficient heating of the at least one aerosol-generating element and prevents the aerosol-generating device being any larger than needed.

The at least one heating article may have any shape. The at least one heating article may have the shape of a bead, capsule, plug, or tablet.

The at least one heating article may have a shape in the form of at least one of a sphere, a cylinder, or a toroid.

The at least one heating article may have a cross section shape which corresponds to the cross sectional shape of the heating chamber. This may allow the at least one heating article to be securely retained in the heating chamber such that it cannot move around within the chamber. This may advantageously improve heat transfer from the at least one heating article to the at least one aerosol-generating element since it minimises air pockets within the heating chamber. This may also prevent multiple heating articles from moving past each other or becoming mixed up inside the heating chamber. This may be advantageous where a user wishes to achieve a customised user experience by arranging a plurality of different heating articles in a specific order. Heating articles configured to release a specific amount of heat may be arranged in the heating chamber adjacent corresponding aerosol-generating elements in the consumable chamber. This may advantageously allow a user to fully customise their user experience by allowing them to use their selected aerosol-generating element with the corresponding heating article.

Where the at least one heating article is spherical or cylindrical in shape, the heating chamber may be generally cylindrical and may have a diameter which is slightly larger than the diameter of the at least one heating article. Where the at least one heating article is toroidal in shape, the heating chamber may also have the general shape of a toroid. Where the heating chamber is configured to receive more than one heating article, the heating chamber may be a toroidal cylinder.

The at least one heating article may have an equivalent diameter of at least about 0.5 millimetres.

The term “equivalent diameter” is used herein to denote the diameter of the sphere which has the same volume as the at least one heating article regardless of the shape of the at least one heating article. As described above, the at least one heating article may have any shape. For a heating article having a spherical shape and a circular transverse cross-section, the equivalent diameter is the diameter of the cross-section of the at least one heating article.

The at least one heating article may have an equivalent diameter of at least about 1 millimetre, at least about 2 millimetres, or at least about 3 millimetres.

The at least one heating article may have an equivalent diameter of less than or equal to about 8 millimetres, less than or equal to about 6 millimetres, or less than or equal to about 5 millimetres.

The at least one heating article may have an equivalent diameter from about 0.5 millimetres to about 8 millimetres, from about 1 millimetre to about 8 millimetres, from about 2 millimetres to about 8 millimetres, or from about 3 millimetres to about 8 millimetres.

The at least one heating article may have an equivalent diameter of about 4 millimetres or about 4.5 millimetres.

The at least one heating article may have a larger equivalent diameter. For example, the at least one heating article may have an equivalent diameter or at least about 5 millimetres, at least about 7 millimetres, or at least about 10 millimetres.

The heating chamber may have a height which is n times the height of a single heating article, where n is an integer. For example, the heating chamber may have a height which is 1, 2, 3, 4, 5, or 6 times the height of a single heating article. This provision prevents any excess space in the heating chamber when the heating chamber is full of heating article. This advantageously provides more efficient heating of the aerosol-generating elements and prevents the aerosol-generating device being any larger than needed.

The at least one heating article and the at least aerosol-generating element may have substantially the same height.

As used herein, the term “height” refers to the dimension of a component of the aerosol-generating system or the kit of parts along the longitudinal axis of the aerosol-generating device. The height of the at least one heating article and the at least one aerosol-generating element is the dimension of these components along the longitudinal axis of the aerosol-generating device when they are received in the heating chamber and the consumable chamber respectively.

The provision of the at least one heating article having substantially the same height as the at least one aerosol-generating element may allow the upstream end of the at least one heating article to be substantially aligned with the upstream end of the at least one aerosol-generating element when the at least one heating article is received in the heating chamber and the at least one aerosol-generating element is received in the consumable chamber. In addition, this arrangement may also allow the downstream end of the at least one heating article to be substantially aligned with the downstream end of the at least one aerosol-generating element when the at least one heating article is received in the heating chamber and the at least one aerosol-generating element is received in the consumable chamber.

This provision may allow for the most efficient heating of the at least one aerosol-generating element by the at least one heating article since the at least one aerosol-generating element is heated along its entire length by the at least one heating article, but the at least one heating article is not generating heat at a location where heat is not required.

The provision of the at least one heating article having substantially the same height as the at least one aerosol-generating element may make it easier for a user to customise their experience by stacking the desired combination and order of aerosol-generating elements in the consumable chamber and stacking the corresponding heating articles in the heating chamber. In use, the first heating article will heat the first aerosol-generating element, the second heating article will heat the second aerosol-generating element and so on. In this way, each aerosol-generating element may be heated by a corresponding heating article which may be configured to provide the appropriate heat for the adjacent aerosol-generating element.

The aerosol-generating system or the kit of parts may comprise a plurality of heating articles and a plurality of aerosol-generating elements.

For example, the aerosol-generating system or the kit of parts may comprise 2, 3, 4, 5, 6, 7, 8, 9, 10, or more heating articles. The aerosol-generating system or the kit of parts may comprise 2, 3, 4, 5, 6, 7, 8, 9, 10, or more aerosol-generating elements.

The aerosol-generating system or the kit of parts may comprise the same number of heating articles as aerosol-generating elements.

This may advantageously allow each aerosol-generating element to be heated by a corresponding heating article which may be configured to provide the appropriate heat for that aerosol-generating element.

The at least one heating article may be configured to raise the temperature of the at least one aerosol-generating element to any temperature. For example, the at least one heating article may be configured to raise the temperature of the at least one aerosol-generating element to between 130 degrees Celsius and 350 degrees Celsius.

The at least one heating article may be configured to maintain the raised temperature for a period of between about 8 minutes and about 10 minutes.

The at least one heating article may be configured to generate heat by an exothermic chemical or physical change.

As described above, the provision of an at least one heating article configured to generate heat by an exothermic chemical or physical change may allow for the aerosol-generating device to generate heat without the need for any electronic components such as electrical heaters or batteries. This may advantageously mean that the aerosol-generating device does not need to be charged from an external power source which may not be generated from a renewable source.

The at least one heating article may be one in which heat is generated by an exothermic chemical change. Where this is the case, two or more chemical reagents may combine and chemically react to form one or more reaction products, the reaction generating heat. Alternatively or in addition, a single reagent may decompose to form a plurality of reaction products, the reaction generating heat.

The at least one heating article may be one in which heat is generated by an exothermic physical change. The physical change may involve one or more substances changing from a liquid state to a solid state, or from a solid state to a liquid state. Alternatively or in addition, the physical change may involve one or more substances changing from a first solid state to a second solid state, or from a first liquid state to a second liquid state. Where this is the case, heating article may comprise a phase change material.

The at least one heating article may comprise at least one of iron and sodium acetate.

Where the at least one heating article comprises iron, the iron may react chemically with oxygen in the air to form iron oxide. This oxidisation reaction may generate heat energy which is used to heat the at least one aerosol-generating element. The iron may comprise iron powder or iron filings. This may increase the surface area of the iron which may advantageously increase heat generated by the at least one heating article.

The at least one heating article may comprise a phase change material. For example, the at least one heating article may comprise sodium acetate. Where the at least one heating article comprises sodium acetate, the at least one heating article may comprise a supersaturated solution of sodium acetate dissolved in a solvent. By disturbing or agitating the supersaturated solution of sodium acetate, the sodium acetate crystallises to form a solid crystal. The physical change releases heat which is used to heat the at least one aerosol-generating element.

The at least one heating article may comprise a supersaturated solution of sodium acetate encapsulated in a polymeric container. The polymeric container may be deformable such that a user may be able to initiate the exothermic crystallisation sodium acetate by disturbing or agitating the polymeric container.

The at least one heating article may comprise a first heating article and a second heating article which, when combined, generate heat.

Where this is the case, the first heating article may comprise a first reagent and the second heating article comprises a second reagent. The first and second heating articles may be kept apart until such time as heat generation is needed, at which point they may be combined to initiate the exothermic chemical reaction to generate heat which is used to heat the at least one aerosol-generating element.

The first heating article may comprise water, and the second heating article may comprise at least one of calcium oxide, calcium chloride, and magnesium iron alloy.

Each of calcium oxide, calcium chloride, and magnesium iron alloy react with water in exothermic reactions to generate heat which is used to heat the at least one aerosol-generating element.

The exothermic chemical or physical change may be a reversible chemical or physical change such that the at least one heating article may be used multiple times.

The provision of an at least one heating article which generates heat using a reversible process may advantageously allow the at least one heating article to be used more than once, reducing waste.

The exothermic chemical or physical change may be reversed by applying energy to the at least one heating article. For example, the exothermic or physical change may be reversed by heating the at least one heating article or by irradiating the at least one heating article, for example with microwave radiation, by immersion in hot liquid, or by placing the at least one heating article in an oven.

To reverse the chemical or physical change, the at least one heating article may be removed from the aerosol-generating device through the opening in the heating chamber. Alternatively, the chemical or physical change may be reversed while the at least one heating article remains within the heating chamber. Where this is the case, the entire aerosol-generating device may be heated or irradiated to reverse the chemical or physical change. In examples where the chemical or physical change may be reversed while the at least one heating article remains within the heating chamber, the heating chamber may not include an opening at all.

The exothermic chemical or physical change may be initiated by applying an electric current to the at least one heating article.

As described above, the activation energy for the exothermic chemical or physical change in the at least one heating article may be provided by an electric current. This electric current may be provided by an electrical power source and electrical circuitry in the aerosol-generating device. In use, a user may activate the electric current when heat from the at least one heating article is required. The electric current may initiate the exothermic chemical or physical change in the at least one heating article.

The exothermic chemical or physical change may be initiated by heating the at least one heating article.

As described above, the activation energy for the exothermic chemical or physical change in the at least one heating article may be provided by an electric heating element. This heating element may be connected to an electrical power source and electrical circuitry in the aerosol-generating device. In use, a user may activate the heating element when heat from the at least one heating article is required. Heat from the heating element may initiate the exothermic chemical or physical change in the at least one heating article.

The exothermic chemical or physical change may be initiated by mechanically agitating the at least one heating article.

As described above, some exothermic chemical or physical changes may be activated by mechanically deforming, agitating, or disturbing the at least one heating article. For example, the crystallisation phase change of a supersaturated solution of sodium acetate may be initiated by mechanically agitating the solution. The at least one heating article may be activated by shaking, cracking, or squeezing the at least one heating article.

The provision of at least one heating article in which an exothermic chemical or physical change may be initiated by mechanically agitation may be advantageous since it may remove the need for an electrical power source and electrical circuitry, simplifying the aerosol-generating device.

Where the exothermic chemical or physical change is initiated by mechanically agitating the at least one heating article, the at least one heating article may further comprise a deformable initiator element, the deformation of which may initiate the exothermic chemical or physical change. For example, where the at least one heating article comprises a supersaturated solution of sodium acetate dissolved in a solvent, a metallic element, such as a metallic disk, may be included in the solution to act as an initiator element. In use, when the metallic element is deformed by a user, nucleation sites are created which initiate the crystallisation of the sodium acetate.

The at least one aerosol-generating element may comprise a solid continuous matrix structure and an aerosol-generating formulation dispersed within the solid continuous matrix structure. The aerosol-generating formulation may be trapped within the solid continuous matrix structure and releasable from the solid continuous matrix structure upon heating of the aerosol-generating element. The solid continuous matrix structure may be a polymer matrix comprising one or more matrix-forming polymers. The aerosol-generating formulation dispersed within the solid continuous matrix structure may comprise at least one alkaloid or cannabinoid compound. The aerosol-generating formulation dispersed within the solid continuous matrix structure may comprise a polyhydric alcohol. The aerosol-generating formulation dispersed within the solid continuous matrix structure may account for at least about 80 percent by weight of a total weight of the aerosol-generating element.

In an aerosol-generating element in accordance with the present invention the solid continuous matrix structure is a polymer matrix comprising one or more matrix-forming polymers. Further, the aerosol-generating formulation dispersed within the solid continuous matrix structure accounts for at least about 80 percent by weight of a total weight of the aerosol-generating element.

The polymer-based solid continuous matrix of aerosol-generating articles in accordance with the present invention provides an inert encapsulation structure for retaining and immobilising the aerosol-generating formulation, which is stable upon heating of the aerosol-generating element during use. The inventors have found that, when heated to temperatures in the range from 150 degrees Celsius to 350 degrees Celsius, aerosol-generating elements in accordance with the present invention release an aerosol as they undergo a significant weight loss. This weight loss is not, however, accompanied by an equally significant volume loss. Without wishing to be bound by theory, it is understood that, upon heating, components of the aerosol-generating formulation originally dispersed and trapped within the solid continuous matrix structure are substantially vaporised and released. On the other hand, components of the solid continuous matrix are substantially unaffected and the solid continuous matrix only partially shrinks while essentially retaining its 3D structure. As such, the encapsulation of the aerosol-generating formulation within the polymer-based matrix advantageously provides minimal or no adverse effects on the sensory profile of the aerosol generated upon heating.

The aerosol-generating element has been found to advantageously provide a controlled delivery of aerosol. Furthermore, the aerosol delivery profile can be readily adjusted by adjusting parameters of the aerosol-generating element such as the size, shape, structure and formulation of the aerosol-generating element.

The at least one aerosol-generating element may be in the form of a discrete, self-standing solid object which is sufficiently stable and robust that it can readily be processed and introduced into an aerosol-generating article using existing methods and techniques.

The at least one aerosol-generating element may be prepared from a matrix precursor solution and components of an aerosol-generating formulation. By way of example, in a method of manufacturing an aerosol-generating element in accordance with the invention, a matrix precursor solution may be provided that comprises a matrix-forming polymer in water. The matrix polymer solution may comprise at least about 35 percent by weight of water, more preferably at least about 40 percent by weight of water. This level of water ensures that the matrix-forming polymer is sufficiently dissolved so that a homogeneous solution is provided.

The invention is defined in the claims. However, below there is provided a non-exhaustive list of non-limiting examples. Any one or more of the features of these examples may be combined with any one or more features of another example, embodiment, or aspect described herein.

Example 1: An aerosol-generating device comprising: a mouthpiece, a heating chamber for receiving at least one heating article, and a consumable chamber for receiving at least one aerosol-generating element, the consumable chamber being in fluid communication with the mouthpiece, the consumable chamber comprising an opening and a closure movable between an open position in which the at least one aerosol-generating element may be inserted into or removed from the consumable chamber through the opening, and a closed position in which the at least one aerosol-generating element is retained in the consumable chamber.

Example 2: An aerosol-generating device according to Example 1, wherein the heating chamber and the consumable chamber are isolated from each other such that heating chamber is not in fluid communication with the consumable chamber.

Example 3: An aerosol-generating device according to Example 1 or Example 2, wherein the heating chamber includes an opening such that at least one heating article may be inserted into and removed from the heating chamber.

Example 4: An aerosol-generating device according to Example 3, wherein the closure of the consumable chamber also closes the opening of the heating chamber, such that when the closure is in the open position at least one heating article may be inserted into or removed from the heating chamber, and when the closure is in the closed position, the at least one heating article is retained in the heating chamber.

Example 5: An aerosol-generating device according to any preceding Example, wherein the closure comprises the mouthpiece.

Example 6: An aerosol-generating device according to any preceding Example, further comprising a heat-conducting element disposed between the heating chamber and the consumable chamber.

Example 7: An aerosol-generating device according to any preceding Example, further comprising an external housing, wherein the external housing is heat insulating.

Example 8: An aerosol-generating device according to any preceding Example, wherein the opening of the consumable chamber is located at the downstream end of the consumable chamber.

Example 9: An aerosol-generating device according to Example 3 or Example 4, wherein the opening of the heating chamber is located at the downstream end of the heating chamber.

Example 10: An aerosol-generating device according to any preceding Example, wherein the heating chamber comprises a longitudinal opening in which the consumable chamber is disposed.

Example 11: An aerosol-generating device according to any preceding Example, wherein the heating chamber is toroidal in shape.

Example 12: An aerosol-generating device according to any preceding Example, wherein the consumable chamber comprises a longitudinal opening in which the heating chamber is disposed.

Example 13: An aerosol-generating device according to any preceding Example, wherein the consumable chamber is toroidal in shape.

Example 14: An aerosol-generating device according to any preceding Example, wherein the heating chamber is formed from a resiliently deformable material.

Example 15: An aerosol-generating device according to any preceding Example, wherein the heating chamber is configured to receive at least one heating article which is configured to generate heat by an exothermic chemical or physical change.

Example 16: An aerosol-generating device according to any preceding Example, wherein the heating chamber is configured to receive at least one heating article which generates heat without electrical power.

Example 17: An aerosol-generating device according to any preceding Example, wherein the heating chamber is divided into a first heating chamber section and a second heating chamber section by a heating chamber barrier, such that a first heating article disposed in the first heating chamber section is kept apart from a second heating article disposed in the second heating chamber section.

Example 18: An aerosol-generating device according to Example 17, wherein the heating chamber barrier is movable between a closed position in which the first heating chamber section and a second heating chamber section are separated such that a first heating article disposed in the first heating chamber section is kept apart from a second heating article disposed in the second heating chamber section, and an open position in which the first heating article disposed in the first heating chamber section is able to mix with the second heating article disposed in the second heating chamber section.

Example 19: An aerosol-generating device according to any preceding Example, further comprising an electrical power source and electrical circuitry configured to initiate an exothermic chemical or physical change in at least one heating article.

Example 20: An aerosol-generating device according to Example 19, wherein the electrical circuitry comprises a heating element configured to heat at least one heating article disposed in the heating chamber.

Example 21: An aerosol-generating device according to any preceding Example, wherein the heating chamber comprises a gas outlet.

Example 22: An aerosol-generating system comprising, an aerosol-generating device according to any preceding Example, at least one heating article disposed in the heating chamber, and at least one aerosol-generating element disposed in the consumable chamber.

Example 23: An aerosol-generating system according to Example 22, wherein the at least one aerosol-generating element has a shape in the form of at least one of a sphere, a cylinder, or a toroid.

Example 24: An aerosol-generating system according to Example 22 or Example 23, wherein the at least one heating article has a shape in the form of at least one of a sphere, a cylinder, or a toroid.

Example 25: An aerosol-generating system according to any one of Examples 22 to 24, wherein the at least one heating article and the at least aerosol-generating element have substantially the same height.

Example 26: An aerosol-generating system according any one of Examples 22 to 25, comprising a plurality of heating articles and a plurality of aerosol-generating elements.

Example 27: An aerosol-generating system according to Example 26, comprising the same number of heating articles as aerosol-generating elements.

Example 28: An aerosol-generating system according to any one of Examples 22 to 27, wherein the at least one heating article is configured to generate heat by an exothermic chemical or physical change.

Example 29: An aerosol-generating system according to Example 28, wherein the at least one heating article comprises at least one of iron and sodium acetate.

Example 30: An aerosol-generating system according to Example 28 or Example 29, wherein the at least one heating article comprises a first heating article and a second heating article which, when combined, generate heat.

Example 31: An aerosol-generating system according to Example 30, wherein the first heating article comprises water, and the second heating article comprises at least one of calcium oxide, calcium chloride, and magnesium iron alloy.

Example 32: An aerosol-generating system according to any one of Examples 28 to 31, wherein the at least one heating article comprises a phase change material.

Example 33: An aerosol-generating system according to any one of Examples 28 to 32, wherein the exothermic chemical or physical change is a reversible chemical or physical change such that the at least one heating article may be used multiple times.

Example 34: An aerosol-generating system according to any one of Examples 28 to 33, wherein the exothermic chemical or physical change is initiated by applying an electric current to the at least one heating article.

Example 35: An aerosol-generating system according to any one of Examples 28 to 33, wherein the exothermic chemical or physical change is initiated by heating the at least one heating article.

Example 36: An aerosol-generating system according to any one of Examples 28 to 33, wherein the exothermic chemical or physical change is initiated by mechanically agitating the at least one heating article.

Example 37: A kit of parts comprising, an aerosol-generating device according to any one of Examples 1 to 21, at least one heating article sized to be received in the heating chamber, and at least one aerosol-generating element sized to be received in the consumable chamber.

Example 38: A kit of parts according to Example 37, wherein the at least one aerosol-generating element has a shape in the form of at least one of a sphere, a cylinder, or a toroid.

Example 39: A kit of parts according to Example 37 or Example 38, wherein the at least one heating article has a shape in the form of at least one of a sphere, a cylinder, or a toroid.

Example 40: A kit of parts according to any one of Examples 37 to 39, wherein the at least one heating article and the at least aerosol-generating element have substantially the same height.

Example 41: A kit of parts according to any one of Examples 37 to 40, comprising a plurality of heating articles and a plurality of aerosol-generating elements.

Example 42: A kit of parts according to Example 41, comprising the same number of heating articles as aerosol-generating elements.

Example 43: A kit of parts according to any one of Examples 37 to 42, wherein the at least one heating article is configured to generate heat by an exothermic chemical or physical change.

Example 44: A kit of parts according to Example 43, wherein the at least one heating article comprises at least one of iron and sodium acetate.

Example 45: A kit of parts according to Example 43 or Example 44, wherein the at least one heating article comprises a first heating article and a second heating article which, when combined, generate heat.

Example 46: A kit of parts according to Example 45, wherein the first heating article comprises water, and the second heating article comprises at least one of calcium oxide, calcium chloride, and magnesium iron alloy.

Example 47: A kit of parts according to any one of Examples 43 to 46, wherein the at least one heating article comprises a phase change material.

Example 48: A kit of parts according to any one of Examples 43 to 47, wherein the exothermic chemical or physical change is a reversible chemical or physical change such that the at least one heating article may be used multiple times.

Example 49: A kit of parts according to any one of Examples 43 to 48, wherein the exothermic chemical or physical change is initiated by applying an electric current to the at least one heating article.

Example 50: A kit of parts according to any one of Examples 43 to 48, wherein the exothermic chemical or physical change is initiated by heating the at least one heating article.

Example 51: A kit of parts according to any one of Examples 43 to 48, wherein the exothermic chemical or physical change is initiated by mechanically agitating the at least one heating article.

Examples will now be further described with reference to the figures in which:

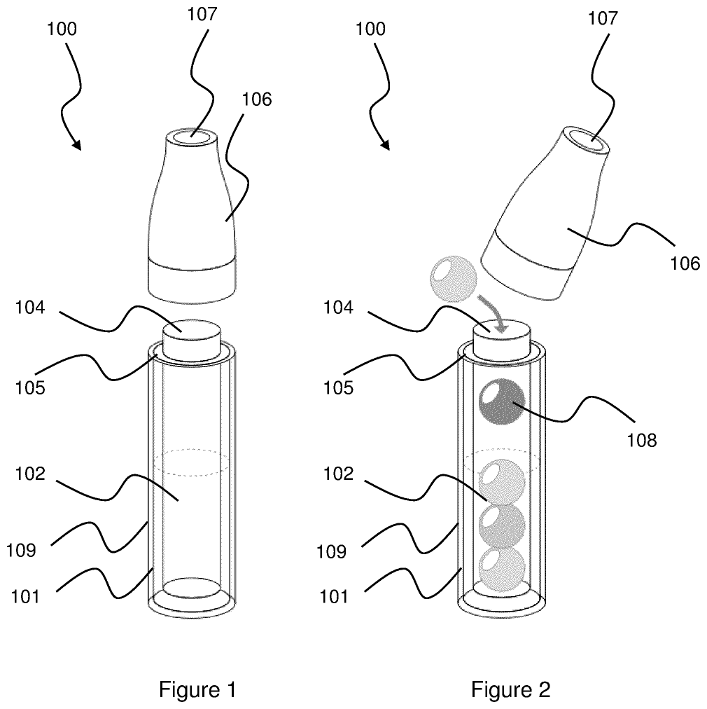

FIG. 1 is a perspective view of a first aerosol-generating device in accordance with the present invention;

FIG. 2 is a perspective view of a first aerosol-generating system in accordance with the present invention;

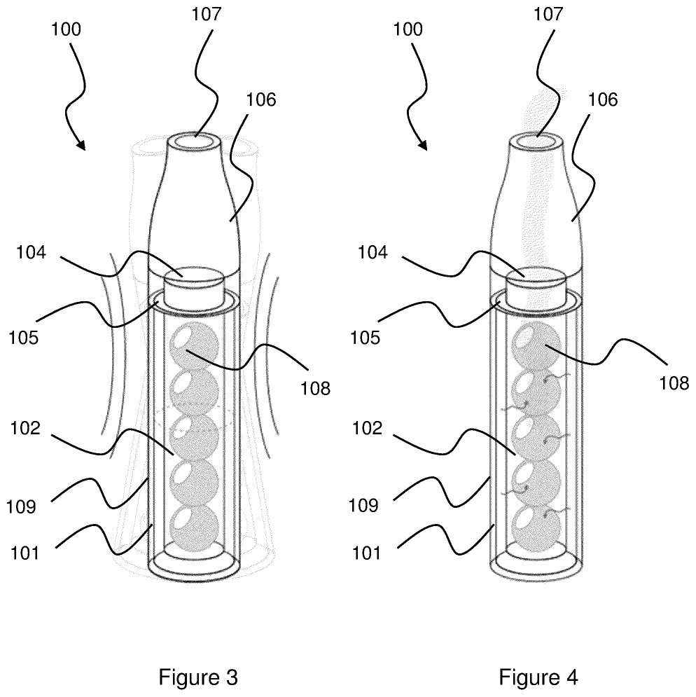

FIG. 3 is a perspective view of a first aerosol-generating system in accordance with the present invention;

FIG. 4 is a perspective view of a first aerosol-generating system in accordance with the present invention;

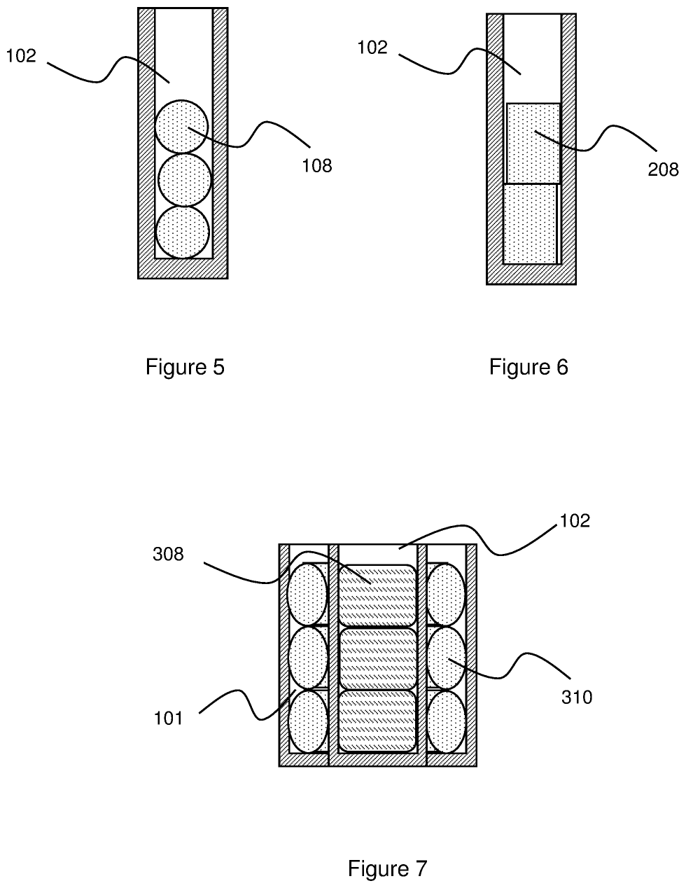

FIG. 5 is a sectional view of a plurality of aerosol-generating elements received in the consumable chamber of a second aerosol-generating device in accordance with the present invention;

FIG. 6 is a sectional view of a plurality of aerosol-generating elements received in the consumable chamber of a third aerosol-generating device in accordance with the present invention;

FIG. 7 is a sectional view of a plurality of aerosol-generating elements received in the consumable chamber, and of a plurality of heating articles received in the heating chamber of a fourth aerosol-generating device in accordance with the present invention.

FIGS. 1 to 4 illustrate the aerosol-generating device of the present invention and the aerosol-generating system of the present invention at four different stages of use.

Each of FIGS. 1 to 4 depict an aerosol-generating device 100. The aerosol generating device 100 comprises a heating chamber 101 for receiving at least one heating article (not shown), and a consumable chamber 102 for receiving at least one aerosol-generating element 108. The heating chamber 101 comprises a heating chamber opening 105 at the downstream end of the heating chamber 101 to allow heating articles to be inserted into and removed from the heating chamber 101. The consumable chamber 102 comprises a consumable chamber opening 104 at the downstream end of the consumable chamber 102 to allow aerosol-generating elements 108 to be inserted into and removed from the consumable chamber 102.

The aerosol-generating device 100 further comprises a closure 106. The closure 106 is movable between an open position, shown in FIGS. 1 and 2, and a closed position, shown in FIGS. 3 and 4. When the closure 106 is in the open position, aerosol-generating elements 108 may be inserted into and removed from the consumable chamber 102 through the consumable chamber opening 104, and heating articles may be inserted into and removed from the heating chamber 101 through the heating chamber opening 105. When the closure 106 is in the closed position, aerosol-generating elements 108 and heating articles are securely retained in the consumable chamber 102 and heating chamber 101 respectively. The closure is 106 is fully detachable from the remainder of the aerosol-generating device 100 in the open position.

The closure 106 includes an airflow passage 107 which extends from the upstream end of the closure 106 to the downstream end of the closure 106. When the closure 106 is in the closed position, the downstream end of the closure 106 is adjacent the opening 104 of the consumable chamber 102. In this way, the opening 104 of the consumable chamber 102 is in fluid communication with the airflow passage 107 when the closure 106 is in the closed position such that air may pass from the consumable chamber 102, through the airflow passage 107, and out of the aerosol-generating device 100. In this way, the closure 106 comprises the mouthpiece of the aerosol-generating device.

The consumable chamber 102 is fully isolated from the heating chamber 101 such that air is unable to pass from the consumable chamber 102 to the heating chamber 101 when in use. The consumable chamber 102 comprises a layer of copper foil (not shown) disposed between the consumable chamber 102 and the heating chamber 101. The aerosol-generating device 100 further comprises an external housing 109 comprising a polymeric material. The heating chamber 101 further comprises a gas outlet (not shown) at the upstream end of the heating chamber 101.

The consumable chamber 102 is cylindrical in shape and extends between an upstream end and a downstream end, the opening 104 of the consumable chamber 102 being disposed at the downstream end of the consumable chamber.

The heating chamber 101 is in the shape of a toroidal cylinder with the cylindrical consumable chamber 102 disposed in the centre of the heating chamber 101 along the longitudinal axis of the aerosol-generating device 100. In other words, the heating chamber 101 fully surrounds the consumable chamber 102 along the longitudinal axis of the aerosol-generating device. The opening 105 of the heating chamber 101 is also toroidal in shape. The heating chamber 101 and the consumable chamber 102 are substantially the same length.

The heating article to be used with the aerosol-generating device 100 is substantially the same shape as the heating chamber 101 and comprises a supersaturated solution of sodium acetate encapsulated in a polymeric container.

The aerosol-generating elements 108 comprise a solid continuous matrix structure and an aerosol-generating formulation dispersed within the solid continuous matrix structure. The aerosol-generating elements 108 are all the same size and shape. The aerosol-generating elements are spherical in shape. The diameter of the aerosol-generating elements 108 is just slightly lower than the inner diameter of the consumable chamber 102. The consumable chamber 102 is sized to accommodate five aerosol-generating elements 108 in an order which is determined when a user inserts the aerosol-generating elements 108 into the consumable chamber 102.

In use, the closure 106 is removed from the remainder of the aerosol-generating device 100. As shown in FIG. 2, a user is then able to insert up to five aerosol-generating elements 108 into the consumable chamber 102 through the opening 104. A heating article is inserted into the heating chamber 101 through the opening 105. The closure 106 is then moved from the open position to the closed position to securely retain the heating article and the aerosol-generating elements 108.

As shown in FIG. 3, once a user wishes to begin the experience, the aerosol-generating device 100 is shaken or otherwise mechanically agitated. This agitation activates the heating article. In particular, the agitation initiates the crystallisation of the supersaturated sodium acetate. This exothermic process releases heat. As shown in FIG. 4, heat from the heating article warms the five aerosol-generating elements causing the aerosol-generating elements 108 to release an aerosol. The aerosol passes out of the outlet 104 of the consumable chamber 102, through the airflow passage 107 of the closure 106 and out of the device.

Following the user experience, the closure 106 is moved to the open position, shown in FIG. 1 and FIG. 2. The used aerosol-generating elements 108 are removed from the consumable chamber 102 through the opening 104. The used heating article is removed from the heating chamber 101 through the opening 105. The used heating article is then heated by, for example adding it to boiling water, to dissolve the crystallised sodium acetate to form the supersaturated solution again, allowing the heating article to be used again.

A cross sectional view of a portion of the aerosol-generating device 100 is shown in FIG. 5. The aerosol-generating elements are shown disposed inside the consumable chamber 102.

FIG. 6 shows a cross sectional view of a portion of an aerosol-generating device 100. In the FIG. 6 example, the aerosol-generating elements 208 are cylindrical rather than spherical as shown in FIGS. 1 to 4. The cylindrical aerosol-generating elements 208 have a diameter which is slightly lower than the inner diameter of the consumable chamber 102. Each of the cylindrical aerosol-generating elements 208 have the same height.

FIG. 7 shows a cross sectional view of a portion of an aerosol-generating device 100. In the FIG. 7 example, the aerosol-generating elements 308 are cylindrical. In the FIG. 7 example, the heating articles 310 are toroidal in shape and fit inside the heating chamber 101 which has the shape of a toroidal cylinder. Unlike the example of FIGS. 1 to 4, in the FIG. 7 example the aerosol-generating system includes three discrete heating articles 310. Each heating article 310 has similar dimensions but is configured to generate different amounts of heat when activated. The FIG. 7 example includes three aerosol-generating elements 308. Each of the three aerosol-generating elements 308 have similar dimensions. In addition, each of the heating articles 310 has substantially the same height as each of the aerosol-generating elements 308 such that, when the three heating articles 310 are each aligned with a corresponding aerosol-generating element 308. The heating article 310 adjacent each aerosol-generating element 308 is configured to heat that aerosol-generating element 308 to a temperature which is optimal for generating aerosol for that aerosol-generating element 308.

In the aerosol-generating devices 100 depicted in FIGS. 1 to 7 the heating chamber 101 of the aerosol-generating device 100 comprises a longitudinal opening in which the consumable chamber 102 of the aerosol-generating device 100 is disposed. However, it will be appreciated that in other embodiments the consumable chamber of the aerosol-generating device may comprise a longitudinal opening in which the heating chamber of the aerosol-generating device is disposed. For example, in an alternate arrangement, an aerosol-generating device may be provided having substantially the same construction as the aerosol-generating device 100 shown in FIG. 7, but in which the position of the heating chamber 101 and the consumable chamber 102 are reversed such that the heating chamber 101 is disposed within a longitudinal opening in the consumable chamber 102. In such an alternate arrangement, the aerosol-generating elements 308 are toroidal in shape and fit inside the consumable chamber 102, which has the shape of a toroidal cylinder, and the heating articles 310 are cylindrical in shape and fit inside the heating chamber 101, which has the shape of a cylinder. Disposing the heating chamber 101 within the consumable chamber 102 may advantageously ensure that a greater proportion of heat generated in the heating chamber 101 is transferred to the consumable chamber 102.

It will be appreciated that in the embodiments described above, heating articles and aerosol-generating elements having other shapes and sizes may be used in the aerosol-generating devices provided that they are sized to fit within the heating chamber and consumable chamber, respectively.

1.-15. (canceled)

16. An aerosol-generating system, comprising:
an aerosol-generating device comprising:
a mouthpiece,
a heating chamber,
at least one heating article disposed in the heating chamber,
a consumable chamber in fluid communication with the mouthpiece, the consumable chamber comprising an opening and a closure movable between an open position in which a plurality of aerosol-generating elements may be inserted into or removed from the consumable chamber through the opening, and a closed position in which the plurality of aerosol-generating elements are retained in the consumable chamber, and
a plurality of aerosol-generating elements disposed in the consumable chamber.

17. The aerosol-generating system according to claim 16, wherein the heating chamber and the consumable chamber are isolated from each other such that the heating chamber is not in fluid communication with the consumable chamber.

18. The aerosol-generating system according to claim 16, wherein the heating chamber includes an opening such that at least one heating article may be inserted into and removed from the heating chamber.

19. The aerosol-generating system according to claim 18, wherein the closure of the consumable chamber also closes the opening of the heating chamber, such that when the closure is in the open position at least one heating article may be inserted into or removed from the heating chamber, and when the closure is in the closed position, the at least one heating article is retained in the heating chamber.

20. The aerosol-generating system according to claim 16, wherein the closure comprises the mouthpiece.

21. The aerosol-generating system according to claim 16, further comprising a heat-conducting element disposed between the heating chamber and the consumable chamber.

22. The aerosol-generating system according to claim 16, further comprising an external housing, wherein the external housing is heat insulating.

23. The aerosol-generating system according to claim 16, wherein the heating chamber comprises a longitudinal opening in which the consumable chamber is disposed.

24. The aerosol-generating system according to claim 16, wherein the heating chamber is formed from a resiliently deformable material.

25. The aerosol-generating system according to claim 16, wherein the heating chamber is configured to receive at least one heating article, which is configured to generate heat by an exothermic chemical or physical change.

26. The aerosol-generating system according to claim 16, wherein the heating chamber is configured to receive at least one heating article, which generates heat without electrical power.

27. The aerosol-generating system according to claim 16, wherein the heating chamber is divided into a first heating chamber section and a second heating chamber section by a heating chamber barrier, such that a first heating article disposed in the first heating chamber section is kept apart from a second heating article disposed in the second heating chamber section.

28. An aerosol-generating device, comprising:
a mouthpiece;
a consumable chamber configured to receive at least one aerosol-generating element, the consumable chamber being in fluid communication with the mouthpiece, the consumable chamber comprising an opening and a closure movable between an open position in which the at least one aerosol-generating element may be inserted into or removed from the consumable chamber through the opening, and a closed position in which the at least one aerosol-generating element is retained in the consumable chamber; and
a heating chamber configured to receive at least one heating article, the heating chamber being disposed within a longitudinal opening in the consumable chamber.

29. A kit of parts, comprising:
an aerosol-generating device according to claim 28;
at least one heating article sized to be received in the heating chamber; and
at least one aerosol-generating element sized to be received in the consumable chamber.

30. The kit of parts according to claim 29, wherein the at least one heating article is configured to generate heat by an exothermic chemical or physical change.
